(19) United States
(12) Patent Application Publication
(10) Pub. No.: US 2025/0268164 A1
(43) Pub. Date: Aug. 28, 2025

(54) A PLANT BREEDING METHOD UTILIZING POLYPLOID HETEROSIS

(71) Applicant: CHINA NATIONAL RICE RESEARCH INSTITUTE, Hangzhou, Zhejiang (CN)

(72) Inventors: Kejian WANG, Hangzhou, Zhejiang (CN); Chaolei LIU, Hangzhou, Zhejiang (CN)

(21) Appl. No.: 18/261,198

(22) PCT Filed: May 23, 2022
(86) PCT No.: PCT/CN2022/094560

§ 371 Date: Jul. 12, 2023

(30) Foreign Application Priority Data
Jun. 23, 2021 (CN) 202110696444.3

(51) Int. Cl. A01H 1/08; A01H 1/02

(52) U.S. Cl. CPC A01H 1/08; A01H 1/02

(57) ABSTRACT

A plant breeding method includes: utilizing technical means to mutate MiMe key genes, screen lines with heterozygous gene loci, and create heterozygous MiMe material; hybridizing heterozygous MiMe material with other plants, and separating new heterozygous MiMe lines from offspring to achieve strategic purpose of transferring MiMe; screening homozygous MiMe material with genetic background recombination in these gregated offspring of heterozygous MiMe material after self-bred; by means of asexual propagation, expanding reproduction of homozygous MiMe with polyploid heterosis, induce and preserve homozygous MiMe by using Fix plants; double self-bred of homozygous MiMe material resulting in polyploid breeding material with heterosis. The invention is applied to combine polyploid breeding with heterosis, solve the problem that the utilization of polyploid heterosis must rely on polyploid induction and hybrid seed must be produced every season, and provide a simple and efficient plant breeding method of utilizing polyploid heterosis.

### TECHNICAL FIELD

The invention belongs to the field of genetic engineering and crop breeding, specifically involves a plant breeding method utilizing polyploid heterosis.

### BACKGROUND TECHNOLOGY

Heterosis is a common phenomenon in the biological world, which is widely used in crop breeding and production practice. To cultivate hybrid varieties, it needs to firstly select homogenous hybrid parental material, but this process requires multiple generations of stable self-bred and takes a long time. After selecting excellent parental materials, it still needs to hybridize parental material annually to produce a large number of seeds before they can be used in agricultural production. Therefore, there are problems like difficulties in breeding and hybrid seed production, and also high seed costs in crop hybrid varieties.

Currently, MiMe strategies with significant breeding value have been developed in crops. In rice, mutate simultaneously the keymeiosis genes OSD1 (OMISSION OF SECOND DIVISION 1), PAIR1 (HOMOLOGOUS PAIRING ABERRATION IN RICE MEIOSIS 1) and REC8 (RECOMBINANT 8), which can transform the meiosis process into similar mitosis process, thus produce clonal gametes that are completely consistent with the genetic material of the maternal somatic cells. This phenotype of mitosis replacing meiosis is called MiMe (mitosis instead of meiosis) (FIG. 1). In Arabidopsis thaliana, mutate simultaneously the meiosis genes OSD1, REC8 and SPO11-1 to obtain the same results, and the MiMe plant phenotype is obtained. In plants, by modifying their genes, all phenotype that can realize mitosis replacing meiosis belong to MiMe strategy (that is, not limited to mutation of the above three genes). MiMe plants are characterized by producing clonal gametes whose genetic material is exactly the same as that of somatic cell, and their genetic material does not undergo recombination. With each self-bred generation, its chromosome number spontaneously doubles once, making it unusable for plant production. In addition, the current MiMe characteristics cannot be transferred among different lines, resulting in difficulties in establishing MiMe for different lines.

The introduction of MiMe strategy into crops is a key step to achieve apomixis of crops, which is the genetic basis of heterosis of fixed plants. MiMe strategy combines with genome elimination gene MTL or key gene BBM which has heterotopic expression of parthenogenesis, can obtain clonal seeds that has completely consistent genetic material with somatic cells, and complete the breakthrough in apomixis in major crops (FIG. 2), but it is still unable to be utilized in production.

Polyploid plants have advantages in biomass, stress-resistance, disease-resistance, and other aspects, and polyploid breeding is one of the main directions for future crop breeding. Compared with homozygous tetraploid, heterozygous tetraploid shows heterosis, and creating heterozygous polyploid has important breeding value. However, the hybrid combinations of polyploid is more difficult. At present, there is no report on the successful combination of heterosis and polyploid advantage at the same time, and used for production and utilization.

At present, if polyploid advantage and heterosis are simultaneously utilized, it needs to double the plant into polyploid through chemical treatment such as colchicine, screen out plant lines with stable genome, and then conduct hybrid of different polyploids to screen out polyploid combinations with obvious heterosis. If utilized in production, it must rely on hybrid seed production. The production and utilization of polyploid heterosis are limited by tedious breeding, long cycle and high cost of seed production (FIG. 3).

The offspring generated by the MiMe strategy will spontaneously double their genome ploidy for each generation. However, due to the fact that the ploidy of MiMe gametes is twice that of normal plant gametes, hybridization with normal plants generally can not bear fruits normally. Additionally, due to the absence of genetic recombination in MiMe plants, even if hybridization is successful, the genetic information will not be exchanged, which cannot meet the requirements of creating genetic diversity in genetic breeding. Therefore, MiMe strategy cannot effectively transfer between different lines, which limits the application of MiMe strategy in polyploid heterosis breeding.

### SUMMARY OF THE INVENTION

The invention provides a strategy for effectively aggregating heterosis and polyploid advantage, and solves the problem that the utilization of polyploid heterosis must rely on polyploid induction and hybrid seed production of each generation.

The invention is implemented based on the following principles. Although the genes controlling MiMe phenotype are known, and the results of their knockout are easy to understand, the invention innovatively uses MiMe heterozygotes for breeding, and effectively combines heterosis with polyploid breeding. This is because the mutation of the MiMe key gene, which results in heterozygous (non homozygous mutations) at all key gene loci, is the basis of the invention. Because homozygous mutations will produce somatic cell gametes, which cannot be hybridized with normal plants, and homozygous genetic material will not be recombined, so genetic diversity material cannot be obtained. However, genetic recombination is the key for plant breeding, homozygous MiMe cannot achieve this goal. Among them, the following aspects are involved.

The first is to create heterozygous MiMe core lines: Taking rice as an example, select lines with excellent performance, and screen lines with all three genes REC8, OSD1, and PAIR1 that are heterozygous through gene mutation, hybridization, and molecular identification. The genotype is represented by AaBbCc (A, B, and C in the entire text specifically match with the three gene loci REC8, OSD1, and PAIR1 respectively). Due to the existence of allelotypes which function have not lost, the AaBbCc lines cannot show the MiMe phenotype, that is, like normal plants, the number of gametic chromosomes of the AaBbCc lines is halved, and genetic material is recombined. Heterozygous AaBbCc lines forms 8 types of gametes, while normal plant only forms one type of gamete (FIG. 4). The second is the hybrid transfer MiMe strategy: Because MiMe strategy makes the gametes of plants change from meiosis to mitosis, chromosomes are doubled, and MiMe lines (genotype aabbcc) and normal plants (genotype AABBCC) cross sterility. Heterozygous MiMe lines (genotype AaBbCc) can form abc genotype gametes with a probability of ⅛, which can reduce the number of chromosomes by half and undergo recombination (FIG. 4). Due to the absence of genomic ploidy differences, the abc genotype gametes of heterozygous MiMe lines can combine with the ABC genotype gametes produced by normal plants to form seeds and generate new heterozygous MiMe material, thereby achieving the goal of transferring MiMe to different background material (FIG. 5). The third is to establish genetic diversity through self-bred: Regardless of whether the initially created heterozygous MiMe or the new heterozygous MiMe material produced through hybridization, the probability of producing homozygous MiMe (genotype aabbcc) after self-bred is 1/64, and the probability of producing heterozygous MiMe (genotype AaBbCc) is ⅛ (FIG. 5). Among them, the genetic material of homozygous MiMe (genotype aabbcc) is recombined, that is, MiMe material with different genetic diversity can be obtained when the population number is enlarged. The genetic material of heterozygous MiMe (genotype AaBbCc) is also recombined, which can not only be reserved as seeds and transferred to MiMe for use, but also create heterozygous MiMe with diverse genetic backgrounds (FIG. 6). The fourth, fix heterosis and polyploid breeding: Homozygous MiMe with genetic diversity is obtained after self-bred and separation of heterozygous MiMe, and lines with excellent phenotypes are screened out. Homozygous MiMe is heterozygous on the entire genome, and after self-bred, it obtains heterozygous tetraploid seeds. Although the genetic background of heterozygous tetraploid seeds is heterozygous, they meet the requirements of population consistency, stability, and specificity in agricultural production, and can be used for plant polyploid breeding and plant production (FIG. 6). The fifth is the self reproduction of MiMe: The genome ploidy of MiMe offspring will spontaneously double, and through asexual reproduction and Fix induction strategies, the self reproduction of MiMe's homozygous lines is achieved (FIG. 7).

Based on the above principles, the technical proposal of this invention is formed combined with experimental exploration.

The invention provides a plant breeding method utilizing polyploid heterosis, which is characterized by, it comprises the following steps:

S1 Utilize technical means to mutate MiMe key genes in plants, screen and regulate lines with heterozygous gene loci, and create heterozygous MiMe material;

S2 Heterozygous MiMe material is hybridized with other normal plants of the same plant strain, and new heterozygous MiMe lines are separated from the offspring;

S3 Self-bred of heterozygous MiMe material and screening of pure MiMe material with genetic background recombination;

Further include the following steps:

S4 By means of asexual reproduction, expand reproduction of homozygous MiMe with polyploid heterosis, preferably, induce through Fix and preserve homozygous MiMe at the same time; and more specifically, MiMe plants are given Fix induced pollen (Fix refers to the lines that simultaneously knock out PAIR1, REC8, OSD1, and MTL, see reference in patent ZL 201811205889.1) to screen diploid plants in hybridized offspring, which can also maintain MiMe's genome ploidy; And,

S5 Self-bred of homozygous MiMe material obtained in step 4, produce polyploid breeding materia with heterosis.

Preferably, the plants mentioned include monocotyledon and dicotyledon; preferably, the plants mentioned include rice, corn, sorghum, millet, barley, wheat, rye, oats, buckwheat, coix seed, sugarcane, asparagus, bamboo shoot, leek, yam, soybean, potato, pea, mung bean, adzuki bean, broad bean, cowpea, kidney bean, lentil, cranberry bean, chickpea, cassava, sweet potato, rape, cotton, beet, eggplant, peanuts, tea, mint, coffee, sesame, sunflower, castor-oil plant, perilla, safflower, tomato, hot pepper, cucumber, green vegetable, lettuce, spinach, garlic, cabbage, leaf mustard, water bamboo, welsh onion, wax gourd, cucurbita pepo, loofah, Chinese cabbage, radish, onion, watermelon, grape, carrot, cauliflower, pumpkin, tobacco, pasture, elephant grass, pennisetum alopecuroides, sudan grass, orchid, lily, tulip and alfalfa.

In the specific implementation method, the asexual reproduction in step 4 is conducted by tillering bending, stem node culture and anther culture.

In the specific implementation method, the technical means for mutation of MiMe key genes in step 1 can be gene editing technology, mutagenesis technology, such as EMS mutagenesis technology, ionizing radiation mutagenesis technology.

In the preferred implementation method, MiMe key genesin plant are able to transform meiosis of germ cell into similar mitosis, and ultimately achieve gametes consistent with ploidy of somatic chromosome.

MiMe key genes include the first gene, the second gene, and the third gene, among which,

The first gene encodes the protein formed in DNA double strand breaks, and the first gene includes PAIR1, PAIR2, PAIR3, PRD1, PRD2, SPO11-1, SPO11-2, SDS, CRC1, P31^{come}, MTOPVIB, DFO and their homologous genes; the second gene encodes and controls the adhesive protein between sisters chromosomes during meiosis, and the second gene includes REC8 and its homologous genes; the third gene encodes the protein involved in the second division of meiosis, and the third gene includes OSD1, TAM, TDM1, and their homologous genes.

In a specific implementation method, step 1 is to first mutate the plant which individual regulatory gene of MiMe phenotype is heterozygous mutation, then obtain hybrids through hybridization between heterozygous mutant lines, screen plants with each regulatory gene of MiMe phenotype are heterozygous mutation in the hybridized offspring, and create heterozygous MiMe material.

In another specific implementation method, step 1 is to simultaneously target the MiMe key gene through multi gene editing system, screen plants which controlling genes all have heterozygous mutations, and create heterozygous MiMe materials.

In order to meet the needs of agricultural production, step 4 is to select plants with excellent agronomic character performance from homozygous MiMe material;

In the preferred implementation method, step 3 is to retain the new obtained heterozygous MiMe material from the self-bred of heterozygous MiMe material, which can be used to proceed with self-bred and screening for homozygous MiMe material with genetic background recombination.

In the preferred implementation method, after step 5, further screen plant lines that meet the requirements of the Agricultural Production Seed Law for stability, consistency, and specificity.

The invention has the following advantages: The invention innovatively utilizes MiMe heterozygotes for breeding, and effectively combines heterosis with polyploid breeding. Therefore, the MiMe strategy is implemented to transfer between different genetic material; in addition, the process of transferring MiMe strategy is also creating genetic diversity germplasm material, and genetic diversity germplasm resource is the basis of plant breeding. It realizes the effective combination of polyploid breeding and heterosis, shortens the breeding period, and does not need matching experiments. This can save the annual seed production process, reduce costs, and have extremely broad application prospects and great application value.

### BRIEF DESCRIPTION OF THE DRAWINGS

FIG. 1 MiMe (mitosis instead of meiosis) strategy diagram of mitosis replacing meiosis phenotype.

FIG. 2 Diagram of MiMe strategy combined with MTL or BBM to achieve apomixis in crops.

FIG. 3 Diagram of breeding strategy for simultaneous utilization of polyploidy and heterosis.

FIG. 4 Diagram of the process of creating heterozygous MiMe and its gametes of the invention.

FIG. 5 Diagram of the hybrid transfer process of MiMe of the invention.

FIG. 6 Diagram of the whole gene genetic material change for transferring MiMe of the invention.

FIG. 7 Diagram of the self reproduction of MiMe of the invention.

FIG. 8 Phenotype of heterozygous MiMe plant created by the invention and the genotype of its controlling gene.

FIG. 9 Hybridization results of heterozygous MiMe and homozygous MiMe with diploid 93-11 inter breed crossing of the invention. The arrow in the left image refers to sturdy seeds.

FIG. 10 Excellent homozygous MiMe and control selected of the invention. A. Excellent homozygous MiMe and control spike part photos; B. Spike grain number statistics of excellent homozygous MiMe and control; C. Statistics of individual plant yield of excellent homozygous MiMe and control. ** represents significant difference at 0.01 level(t-test).

FIG. 11 Excellent homozygous MiMe and its offspring tetraploid plants screened by the invention. A. Photos of homozygous MiMe and tetraploid plants at heading stage; B. Plant height statistics of homozygous MiMe and tetraploid plants; C. Biomass statistics of homozygous MiMe and tetraploid. ** represents significant difference at 0.01 level(t-test).

FIG. 12 Result of flow cytometry identification of genomic ploidy after Fix induction.

FIG. 13 Figure of spike of tetraploid and diploid and flow cytometry identification result. A. Spike figures of tetraploid and diploid (right and left, respectively); B. Flow cytometry identification results of diploid plant (as control); C. Flow cytometry identification results of tetraploid plant.

FIG. 14 Field planting of tetraploid plants.

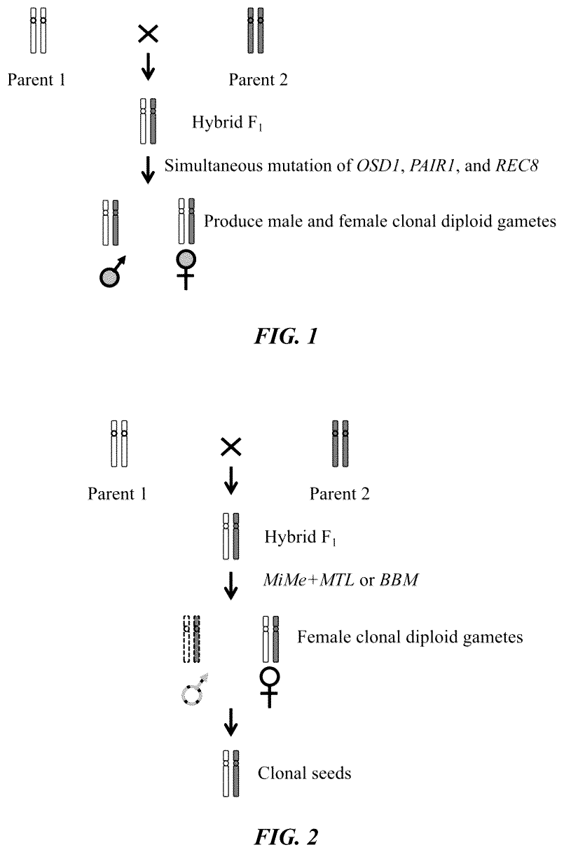

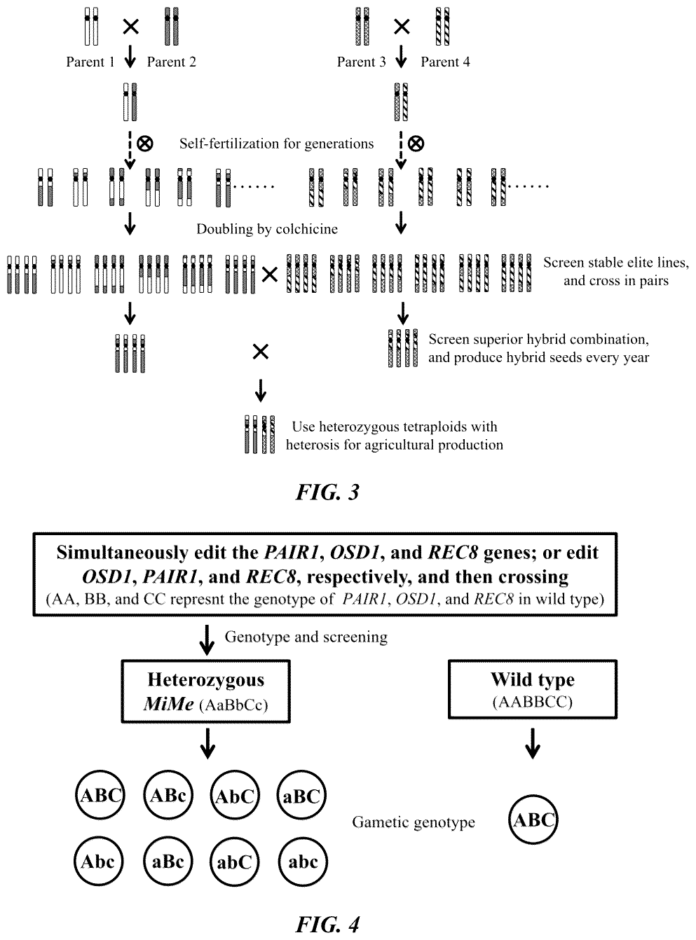

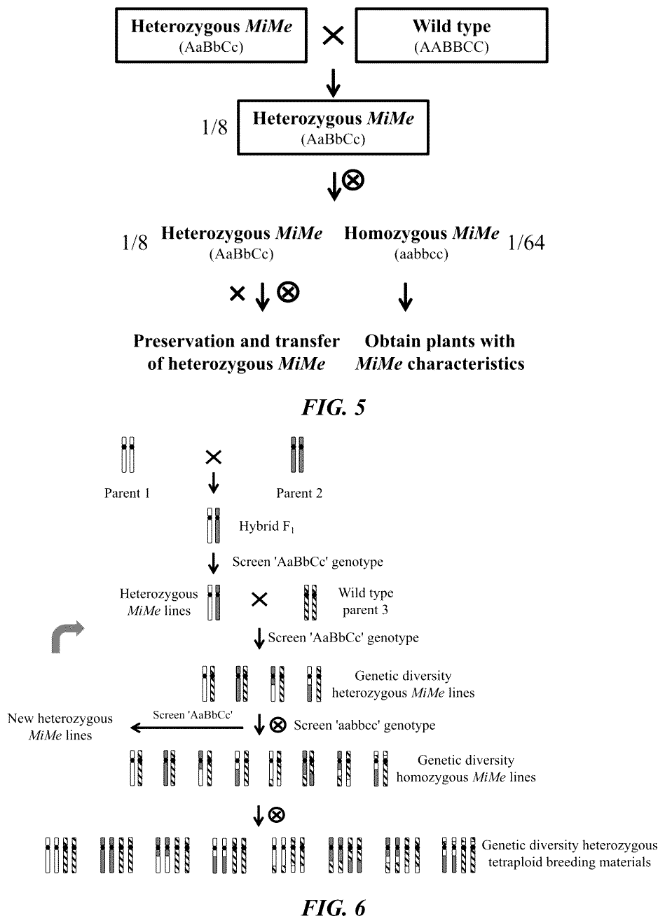

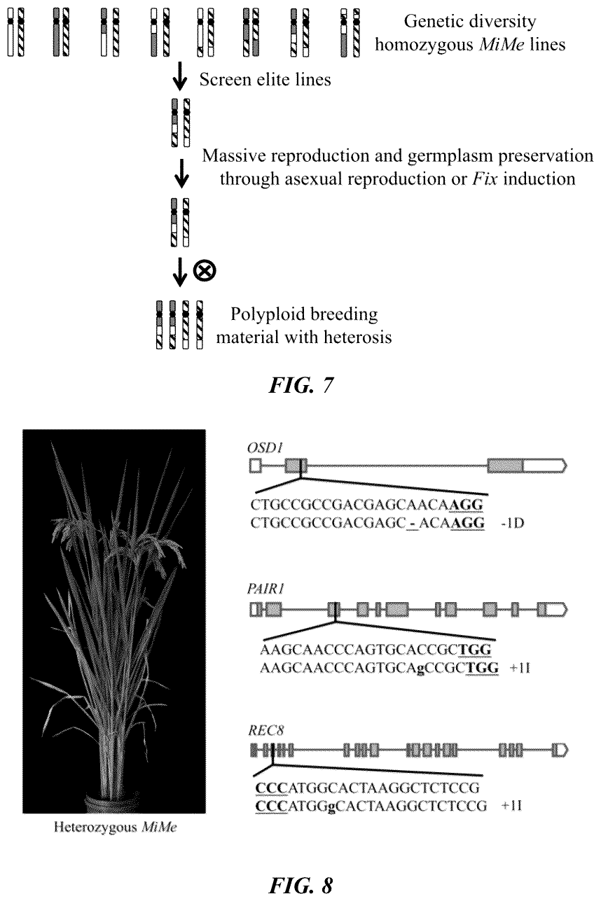

### DETAILED DESCRIPTION OF THE PREFERRED EMBODIMENTS

The invention is further elaborated through specific implementations, but does not constitute any limitation to the invention.

In the following implementations, the indica-japonica hybrid rice Chunyou 84 is used as the genetic transformation material, and its REC8, OSD1 and PAIR1 genes are mutated by genome editing technology. After hybridization, the three gene loci are obtained through molecular identification and screening, which are all heterozygous lines, namely heterozygousMiMe. Then, the heterozygous MiMe material is hybridized with rice varieties such as 93-11, Huazhan, and Wuyunjing No.7, respectively. In the F1generation of crossbreed, molecular markers are used to screen all three gene loci as heterozygous lines, obtaining new heterozygous MiMe and achieving the goal of transferring MiMe. The created heterozygous MiMe is screened for homozygous MiMe utilizing molecular markers after self-bred. The seeds produced by homozygous MiMe self-bred are used for plant polyploid breeding and production.

### Embodiment 1: Construction of Gene Knockout Vector

The main steps are as follows (specific operations can also refer to the methods recorded in CN201510485573.2):

### 1 Construction of SK gRNA

Select the following three loci as the CRISPR-Cas9 gene editing system's knockout loci for REC8, OSD1, and PAIR1 (underline represents PAM sequences):

REC8 gene knockout locus (SEQ ID NO: 1):
CCCATGGCACTAAGGCTCTCCG
OSD1 gene knockout locus(SEQ ID NO: 2):
CTGCCGCCGACGAGCAACAGG
PAIR1 gene knockout locus (SEQ ID NO: 3):
AAGCAACCCAGTGCCACGCTGG

Design two complementary DNA sequences: Adding GGCA before the forward sequence and AAAC before the reverse complementary sequence.

There are two AarI restriction site on SK-gRNA, which are digested by AarI to form vector with sticky ends; after denaturing and annealing the designed target sequence with forward and reverse primers, T4 ligase is connected to the previously constructed intermediate vector SK-gRNA to form a single target gRNA.

### 2 Construction of the Final Binary Expression Vector

SK-gRNA OSD1, SK-gRNA PAIR1, and SK-gRNA REC8 are digested with KpnI and BglII respectively, and fragments are collected and connected to the binary vector pC1300-Cas9 expressing Cas9 protein (between KpnI and BamHI loci). Finally, the REC8, OSD1, and PAIR1 gene knockout vectors pC1300-Cas9-gRNA OSD1 are obtained respectively. pC1300-Cas9gRNA REC8 and pC1300-Cas9-gRNA PAIR1 are used for transgenic rice multiple mutants preparation.

### Embodiment 2: Obtaining Transgenic Plants

The expression vectors pC1300-Cas9-gRNA OSD1, pC1300-Cas9gRNA REC8, and pC1300-Cas9-gRNA PAIR1 are transferred into agrobacterium (AgroBacteriumtumefaciens) strain EHA105 by electric shock, and the binary expression vector is transferred into the callus of rice Chunyou 84 utilizing agrobacterium mediated method. The specific method of transformation is to sterilize the embryos of hybrid rice Chunyou 84 seeds and inoculate them into a culture medium for inducing callus. After one week of cultivation, select embryogenic callus tissue that is vigorous in growth, light yellow in color, and relatively loose, and use it as a receptor for transformation. Rice callus tissues are infected with EHA105 lines containing plasmids of pC1300 Cas9 gRNA OSD1, pC1300 Cas9 gRNA REC8, and pC1300 Cas9 gRNA PAIR1 respectively. After 3 days of cultivation at 25° C. in the dark, resistant callus tissues and transgenic plants are screened on a selective culture medium containing 50 mg/l hygromycin. Select transgenic plants that grow normally on hygromycin selective culture medium.

### Embodiment 3: Sequencing Identification of Three Mutants

Identification of target gene mutations utilizing molecular biology methods. The CTAB method extracts the genome DNA of transgenic plants from individual plants, and PCR amplifies the target band. Primer pairs used:

REC8-F (SEQ ID NO: 4):
gcgacgttcactcgaagatca
REC8-R (SEQ ID NO: 5):
cgccatgcctegtgatctcaa
OSD1-F (SEQ ID NO: 6):
atctccaggatgctgaagtgag
OSD1-R (SEQ ID NO: 7):
cctagactgctatcttgctagtgat
PAIR1-F (SEQ ID NO: 8):
ctgtacctgtgcatctaattacag
PAIR1-R (SEQ ID NO: 9):
ccccacttatgtactgagcttccag

The obtained PCR products are sent to the sequencing company for sequencing using OSD1-F, PAIR1-F, and REC8-F as sequencing primers. The obtained results are compared with wild-type sequences. The sequencing results are bimodal and analyzed using degenerate codon strategy (http://dsdecode.scgene.com/ perform peak plot analysis) to directly obtain heterozygous mutation information.

Screen lines which OSD1, REC8, and PAIR1 are heterozygous for pairwise hybridization, and screen lines with both loci are heterozygous in the hybrid offspring and conduct hybridization again. Screen material with three loci are heterozygous in their offspring. The final heterozygous MiMe is obtained, and the plant and genotype results are shown in FIG. 8.

The lines with heterozygous mutations at all three loci are heterozygous MiMe materials used for transferring MiMe between different lines.

### Embodiment 4: Hybrid Transfer MiMe Strategy

Plant heterozygous MiMe, 93-11, Huazhan, and Wuyunjing No.7 in the field. At the heading stage, hybrid F1 seeds are obtained by crossing heterozygous MiMe with 93-11, Huazhan, and Wuyunjing No.7.

These F1 seeds are germinated in the laboratory, and the genome DNA of hybrid plants is extracted using CTAB method. The target bands are amplified by PCR using primer pairs of REC8-F/R, OSD1-F/R, and PAIR1-F/R. The obtained PCR products are sent to the sequencing company for sequencing using OSD1-F, PAIR1-F, and REC8-F as sequencing primers. In hybrid F1 seeds, screen lines with all three loci are heterozygous mentioned above to obtain new heterozygous MiMe material.

### Embodiment 5: Obtaining Homozygous MiMe Lines through Self-Bred

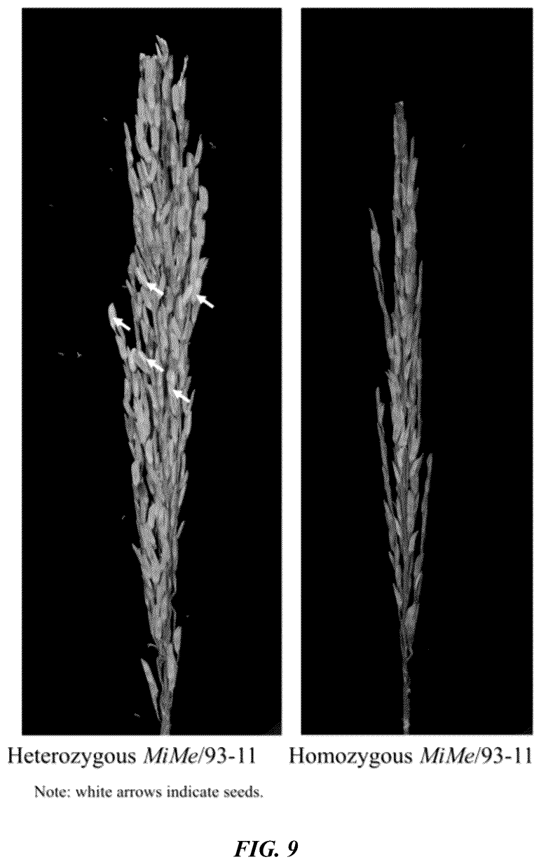

The newly obtained heterozygous MiMe material will produce a certain proportion of homozygous MiMe after self-bred for one generation. After extracting DNA, PCR sequencing are used to identify the three loci mentioned above, and lines with homozygous mutations at all three loci are screened to obtain homozygous MiMe. At the same time, all three loci are preserved as heterozygous MiMe. Hybridizing heterozygous MiMe and homozygous MiMe with 93-11 strain respectively, it is found that hybridization between heterozygous MiMe and 93-11 is fertile, while hybridization between homozygous MiMe and 93-11 is sterile, as shown in FIG. 9.

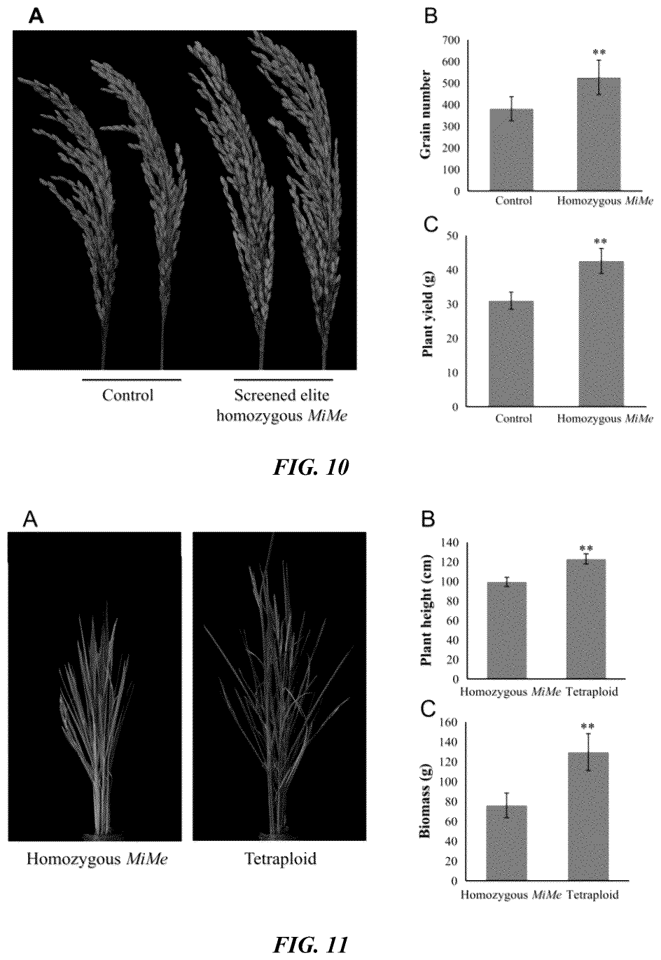

At this point, the genetic background of different homozygous MiMe individuals is inconsistent, and a certain number of homozygous MiMe forms a genetic diversity homozygous MiMe population. For the heterozygous MiMe here, the genetic material between individual plants is also inconsistent, which can be used to self-bred to produce new homozygous MiMe, or cross to produce new heterozygous MiMe. When conducting polyploid breeding, screen homozygous MiMe lines with excellent performance. Compared with the control, the screened homozygous MiMe lines showed large ears, increased number of ears, and increased yield per plant as shown in FIG. 10. Through tillering bending and stem node culture during the tillering stage, homozygous MiMe is propagated and its seeds are harvested at maturity. The seed is tetraploid and heterozygous throughout the genome, but it meets the requirements for consistency, stability, and specificity of the seed and can be used for plant production. Screen excellent diploid plants and their doubled tetraploid plants (see FIG. 11). Compared to homozygous MiMe, the doubled tetraploid plants shows a 20% increase in height and a 50% increase in biomass, indicating a significant advantage.

### Embodiment 6: Propagation of MiMe

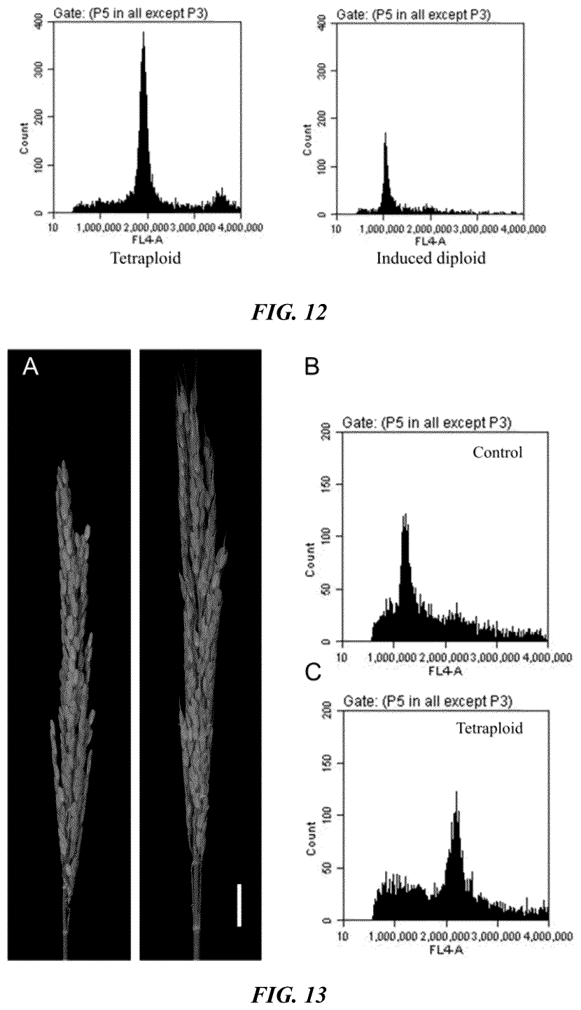

When conducting polyploid breeding, screen homozygous MiMe lines with excellent performance (including biomass, yield, resistance, etc.), and propagate them through asexual reproduction methods such as tillering bending, stem node culture, and anther culture. In addition, grant induced pollen to MiMe plants using Fix (Fix refers to the lines that simultaneously knock out PAIR1, REC8, OSD1, and MTL, see reference in patent ZL 201811205889.1), screen diploid plants in hybrid offspring and can also maintain MiMe genome ploidy. After induction, the genome ploidy is identified using flow cytometry to obtain plants without genome doubling. The ploidy identification results are shown in FIG. 12.

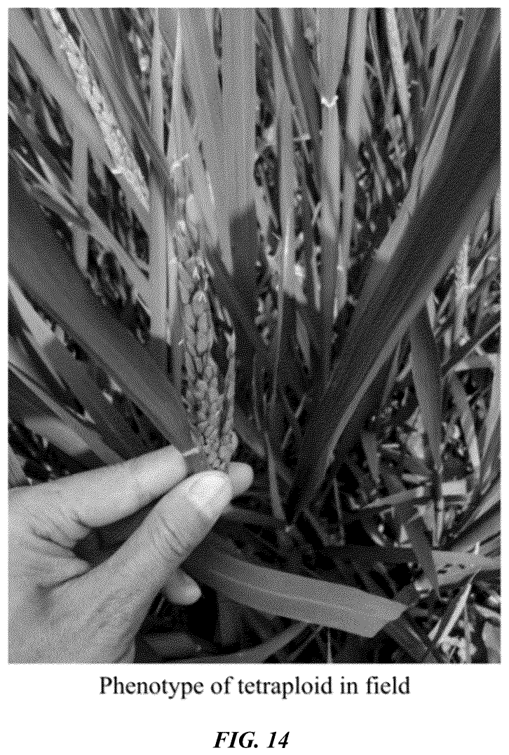

Harvest MiMe seeds at maturity. The seed belongs to tetraploid. After germination and growth into a plant, the tetraploid glume has awns (awning is one of the important indicators of rice tetraploid), and the plant ploidy is identified as tetraploid by flow cytometry, the result is shown in FIG. 13. Planting tetraploid seeds in residential areas has consistent performance as in the fields. (see FIG. 14), which meets the requirements for consistency, stability, and specificity of seeds and can be used for plant production.

1. A plant breeding method utilizing polyploid heterosis, characterized in that, comprising the following steps:
1) utilizing technical means to mutate MiMe key genes in plants, screening lines with heterozygous at regulated gene loci, and creating a heterozygous MiMe material;
2) hybridizing the heterozygous MiMe material with a wild type plant, and obtaining a new heterozygous MiMe line from an offspring;
3) self-breeding the heterozygous MiMe material and screening of a homozygous MiMe material with genetic background recombination;
further comprising following steps:
4) by means of asexual reproduction, expanding reproduction of the homozygous MiMe material with polyploid heterosis, preferably, inducing by using Fix plants and preserve the homozygous MiMe material at the same time; and,
5) self-breeding of the homozygous MiMe material obtained in step 4, producing a polyploid breeding material with heterosis.

2. The method according in claim 1, characterized in that, the plants include monocotyledon and dicotyledon; preferably, the plants include rice, corn, sorghum, millet, barley, wheat, rye, oats, buckwheat, coix seed, sugarcane, asparagus, bamboo shoot, leek, yam, soybean, potato, pea, mung bean, adzuki bean, broad bean, cowpea, kidney bean, lentil, cranberry bean, chickpea, cassava, sweet potato, rape, cotton, beet, eggplant, peanuts, tea, mint, coffee, sesame, sunflower, castor-oil plant, perilla, safflower, tomato, hot pepper, cucumber, green vegetable, lettuce, spinach, garlic, cabbage, leaf mustard, water bamboo, welsh onion, wax gourd, cucurbita pepo, loofah, Chinese cabbage, radish, onion, watermelon, grape, carrot, cauliflower, pumpkin, tobacco, pasture, elephant grass, pennisetum alopecuroides, sudan grass, orchid, lily, tulip and alfalfa.

3. The method written according to claim 1, characterized in that, asexual propagation in step 4 is conducted by tillering bending, stem node culture and anther culture.

4. The method according to claim 1, characterized in that, the technical means for mutation of MiMe key genes in step 1 can be gene editing technology, mutagenesis technology, such as EMS mutagenesis technology, ionizing radiation mutagenesis technology.

5. The method according to claim 1, characterized in that, MiMe key genes in plants are adpated to transform meiosis of germ cell into similar mitosis, and ultimately achieve gametes consistent with ploidy of somatic chromosome.

6. The method according to claim 5, characterized in that, MiMe key genes include a first gene, a second gene and a third gene;
the first gene encodes the protein formed in DNA double strand breaks, and the first gene includes PAIR1, PAIR2, PAIR3, PRD1, PRD2, SPO11-1, SPO11-2, SDS, CRC1, P31^{come}, MTOPVIB, DFO, and their homologous genes;
the second gene encodes and controls the adhesive protein between sisters chromosomes during meiosis, and the second gene includes REC8 and its homologous genes;
the third gene encodes the protein involved in the second division of meiosis, and the third gene includes OSD1, TAM, TDM1, and their homologous genes.

7. The method according to claims 6, characterized in that, step 1 is to first mutate the three regulatory genes of MiMe in heterozygous individually, then obtain hybrids through hybridization between heterozygous mutants, screen plants with each regulatory gene of MiMe are heterozygous mutation in the hybridized offspring, and create heterozygous MiMe material.

8. The method according to claims 6, characterized in that, step 1 is to simultaneously target the MiMe controlling genes through multi gene editing system, screen plants which controlling genes all are heterozygous mutations, and create heterozygous MiMe materials.

9. The method according to claim 1, characterized in that, step 3 is to retain the new obtained heterozygous MiMe material from the self-bred of heterozygous MiMe material, which can be used to proceed with self-bred.

10. The method according to claim 1, characterized in that, step 4 is to select plants with excellent agronomic character performance from homozygous MiMe material; and/or after step 5, further screen lines that meet the requirements of the Agricultural Production SeedLaw for stability, consistency, and specificity.


SEQUENCE LISTING (10 sequences)

SEQ ID NO: 1 - DNA, 22 bp, Artificial Sequence
  REC8 gene knockout locus
cccatggcactaaggctctccg

SEQ ID NO: 2 - DNA, 22 bp, Artificial Sequence
  OSD1 gene knockout locus
ctgccgccgacgagcaacaagg

SEQ ID NO: 3 - DNA, 22 bp, Artificial Sequence
  PAIR1 gene knockout locus
aagcaacccagtgcaccgctgg

SEQ ID NO: 4 - DNA, 22 bp, Artificial Sequence
  REC8-F primer
gcgacgcttcactcgaagatca

SEQ ID NO: 5 - DNA, 22 bp, Artificial Sequence
  REC8-R primer
cgccatgcctcgttgatctcaa

SEQ ID NO: 6 - DNA, 23 bp, Artificial Sequence
  OSD1-F primer
atctccaggatgcctgaagtgag

SEQ ID NO: 7 - DNA, 26 bp, Artificial Sequence
  OSD1-R primer
cctagactgctactcttgctagtgat

SEQ ID NO: 8 - DNA, 24 bp, Artificial Sequence
  PAIR1-F primer
ctgtacctgtgcatctaattacag

SEQ ID NO: 9 - DNA, 22 bp, Artificial Sequence
  PAIR1 gene knockout locus
aagcaacccagtgcaccgctgg

SEQ ID NO: 10 - DNA, 27 bp, Artificial Sequence
  PAIR1-R primer
ccccatcttatgtactgagcttgccag